(19) United States
(12) Patent Application Publication
(10) Pub. No.: US 2009/0142225 A1
(43) Pub. Date: Jun. 4, 2009

(54) Ice Bins, Ice Makers, and Methods for Cleaning Ice Bins and Ice Makers

(75) Inventor: Niklas Tornqvist, Espoo (FI)

Correspondence Address: POTOMAC PATENT GROUP PLLC, P. O. BOX 270, FREDERICKSBURG, VA, 22404, US

(73) Assignee: Biozone Scientific International Oy, Espoo (FI)

(21) Appl. No.: 11/949,577

(22) Filed: Dec. 3, 2007

(51) Int. Cl. A61L 2/20

(52) U.S. Cl. 422/28; 62/78

(57) ABSTRACT

An ice maker includes an ozone generator that has a housing having an air inlet and an air outlet functionally connected to the air inlet for allowing air to flow through the housing. The ozone generator further includes an ozone generating device in the housing. The air outlet is functionally connected to a thermally insulated interior of the ice bin for feeding air containing ozone from ozone generator to the thermally insulated interior, and the air inlet of the ozone generator is functionally connected to the thermally insulated interior of the ice bin for receiving air from the thermally insulated interior. Ice bins and methods for cleaning an ice bin and an ice maker are also described.

### BACKGROUND

The invention relates to storing and the manufacturing of ice, such as ice cubes, and particularly to an ice bin and to a method for cleaning an ice bin and to an ice maker and to a method for cleaning an ice maker.

It is known to use ozone generators in connection with ice bins and ice makers to produce ozone i.e. photo plasma consisting of various ionization stages of water vapor (hydroxyl radicals OH*, superoxide ions, free electrons, etc.), oxygen and combinations of these that all have similar properties to ozone (hereinafter referred to as “ozone”) to purify the thermally insulated interior and possible appliances such as molds for ice cubes arranged in the thermally insulated interior (hereinafter referred to a “thermally insulated interior”) of ice bins and ice makers and to kill microorganisms in the thermally insulated interior of ice bins and ice makers. This has been suggested in, for example:

U.S. Pat. No. 6,167,711 (Slattery et. al),

U.S. Pat. No. 6,334,328 (Brill),

U.S. Pat. No. 6,458,257 (Andrews et. al),

U.S. Pat. No. 6,506,428 (Berge et. al),

U.S. Pat. No. 6,571,573 (Henry),

U.S. Pat. No. 6,967,008 (Barnes),

U.S. Patent Application Publication No. US 2003/0080467 (Andrews et. al), and

U.S. Patent Application Publication No. US 2007/0214809 (DiLorenzo).

### SUMMARY

It is an object of the invention to improve the function of an ozone generator of an ice bin and correspondingly to improve the function of an ozone generator of an ice maker and as a result of this to provide an ice bin and a method for cleaning an ice bin and correspondingly to provide an ice maker and a method for cleaning an ice maker.

An ozone generator comprises generally a housing having an air inlet and an air outlet for allowing air to flow through the housing. The air inlet of the housing is functionally connected to the air outlet of the housing for allowing air to flow through the housing. The ozone generator comprises ozone generating means in the housing for generating ozone in air flowing through the housing. By connecting an ozone generator to an ice bin or an ice maker so that the air outlet of the ozone generator is functionally connected to the thermally insulated interior of the ice bin or the ice maker, air containing ozone can be fed into the thermally insulated interior of the ice bin or the ice maker to eliminate containments in the thermally insulated interior of the ice bin or the ice maker. Ozone is one of the most effective substances in eliminating unwanted micro-organisms, breaking down unwanted chemicals, and getting rid of bad odor. Ozone is therefore commonly used in connection with ice bins or ice makers.

In the previously known apparatuses the air flow through the housing of the ozone generator goes from the outside of the thermally insulated interior of the ice bin or the ice maker into the thermally insulated interior of the ice bin or the ice maker. This leads to the fact that new contaminates such as new microbes and new micro-organisms are constantly drawn from the outside of the thermally insulated interior of the ice bin or the ice maker into the thermally insulated interior of the ice bin or the ice maker via the ozone generator at the same time as the ozone generator is producing ozone for inhibiting the growth of contaminates such as microbes and micro-organisms.

One facility where ice bins or ice makers are commonly used is in kitchens of fast food restaurants. Because in previously known apparatuses air to the ozone generator is drawn from the outside of the thermally insulated interior of the ice bin or the ice maker i.e. from the thermally insulated interior of the restaurant kitchen, where the ice bin or the ice maker is placed, into the thermally insulated interior of the ice bin or the ice maker, a large amount of contaminates present in the air of the restaurant kitchen will end up in the ice cubes and finally be drunk/eaten by the customers, which is a health hazard.

In an ice bin and an ice maker of the invention the air inlet of the ozone generator for introducing air into the thermally insulated interior of the ozone generator is arranged in the thermally insulated interior of the ice bin and the ice maker. In other words, in an ice bin and an ice maker of the invention the air inlet of the ozone generator for introducing air into the thermally insulated interior of the ozone generator is functionally connected to the thermally insulated interior of the ozone generator. This significantly reduces the amount of new contaminates such as new microbes and new micro-organisms introduced into the thermally insulated interior of the ice bin or the ice maker.

In a preferred embodiment of the ice bin the air inlet of the housing of the ozone generator is functionally connected with the thermally insulated interior of the ice maker to receive air solely from the thermally insulated interior of the ice maker.

In a preferred embodiment of the ice maker the air inlet of the housing of the ozone generator is functionally connected with the thermally insulated interior of the ice maker to receive air solely from the thermally insulated interior of the ice maker.

In another preferred embodiment of the ice maker the thermally insulated interior of the ice maker comprises an ice making section comprising ice making means for making ice and an ice storage section for storing ice, the ice storage section being configured to receive ice from the ice making section. In this preferred embodiment of the invention the air inlet of the ozone generator is functionally connected to the ice making section to receive air from the ice making section and the air outlet of the ozone generator is functionally connected to the ice making section for discharging air containing ozone to the ice making section and the ozone generator is arranged in the ice making section of the thermally insulated interior of the ice maker. In this preferred embodiment the air inlet of the housing of is preferably, but not necessarily, functionally connected with the ice making section to receive air solely from the ice making section.

In another preferred embodiment of the ice maker the thermally insulated interior of the ice maker comprises an ice making section comprising ice making means for making ice, and an ice storage section for storing ice, the ice storage section being configured to receive ice from the ice making section. In this preferred embodiment of the invention the air inlet of the ozone generator is functionally connected to the ice storage section and the air outlet of the ozone generator is functionally connected to the ice storage section and the ozone generator is arranged in the ice storage section. In this preferred embodiment the air inlet of the housing is preferably, but not necessarily, functionally connected to the ice storage section to receive air solely from the ice storage section.

In another preferred embodiment of the ice maker the thermally insulated interior of the ice maker comprises an ice making section comprising ice making means for making ice, and an ice storage section for storing ice, the ice storage section being configured to receive ice from the ice making section of the thermally insulated interior of the ice maker. In this preferred embodiment of the invention the ice maker comprises an ozone generator in the form of a first ozone generator and in addition to the first ozone generator a second ozone generator. In this preferred embodiment of the invention a first air inlet of the first ozone generator is functionally connected to the ice storage section and a first air outlet of the first ozone generator is functionally connected to the ice storage section and the first ozone generator. In this preferred embodiment of the invention a second air inlet of the second ozone generator is functionally connected to the ice storage section and a second air outlet of the second ozone generator is functionally connected to the ice storage section. In this preferred embodiment the first air inlet of the first housing of the first ozone generator is preferably, but not necessarily, functionally connected to the ice making section of the thermally insulated interior of the ice maker to receive air solely from the ice making section of the thermally insulated interior of the ice maker. In this preferred embodiment the second air inlet of the housing of the second ozone generator is preferably, but not necessarily, functionally connected with the ice storage section of the thermally insulated interior of the ice maker to receive air solely from the ice storage section of the thermally insulated interior of the ice maker.

### BRIEF DESCRIPTION OF THE DRAWINGS

Next, preferred embodiments of the invention will be described in greater detail by referring to the following figures, in which:

FIG. 1 shows an ice bin,

FIG. 2 is a cross-section view of a first embodiment of an ice bin,

FIG. 3 is a cross-section view of a second embodiment of an ice bin,

FIG. 4 is a cross-section view of a third embodiment of an ice bin,

FIG. 5 shows an ice maker,

FIG. 6 shows an ozone generator,

FIG. 7 shows the ozone generator in FIG. 6 in cross-section view,

FIG. 8 is a cross-section view of a first embodiment of an ice maker in which an air inlet of an ozone generator is functionally connected to a ice making section of an ice maker,

FIG. 9 is a cross-section view of a second embodiment of an ice maker in which an air inlet of an ozone generator is functionally connected to a ice making section of an ice maker and in which the ozone generator is arranged in the ice making section,

FIG. 10 is a cross-section view of a third embodiment of an ice maker in which an air inlet of an ozone generator is functionally connected to a ice storage section of an ice maker,

FIG. 11 is a in cross-section view of a fourth embodiment of an ice maker in which an air inlet of an ozone generator is functionally connected to a ice storage section of an ice maker and in which the ozone generator is arranged in the ice storage section,

FIG. 12 is a cross-section view of a fifth embodiment of an ice maker in which an air inlet of a first ozone generator is functionally connected to a ice making section of an ice maker and in which an air inlet of a second ozone generator is functionally connected to a ice storage section of an ice maker,

FIG. 13 is a cross-section view of a sixth embodiment of an ice maker in which an air inlet of a first ozone generator is functionally connected to a ice making section of an ice maker and in which an air inlet of a second ozone generator is functionally connected to a ice storage section of an ice maker and in which the first ozone generator is arranged in the ice making section and in which the second ozone generator is arranged in the ice storage section,

FIG. 14 is a cross-section view of a seventh embodiment of an ice maker,

FIG. 15 is a cross-section view of a eight embodiment of an ice maker,

FIG. 16 shows in cross-section view a first ozone generator,

FIG. 17 shows in cross-section view a second ozone generator

FIG. 18 is a cross-section view of a tenth embodiment of an ice maker, and

FIG. 19 shows in cross-section view the ozone generator of the ice maker shown in FIG. 17.

### DETAILED DESCRIPTION

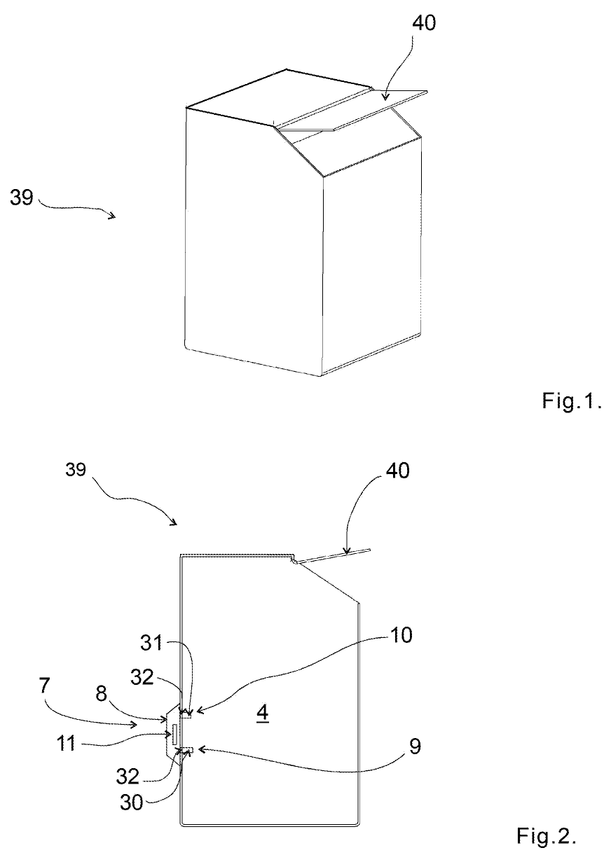

FIG. 1 shows an ice bin 39 for storing ice. The ice bin 39 comprises an ozone generator 7 for generating ozone. The ozone generator 7 comprises a housing 8 having an air inlet 9 and an air outlet 10. The air inlet 9 of the housing 8 of the ozone generator 7 is functionally connected to the air outlet 10 of the housing 8 of the ozone generator 7 for allowing air to flow through the housing 8. The ozone generator 7 comprises ozone generating means 11 in the housing 8 for generating ozone in air flowing through the housing 8. The ozone generating means 11 is preferably, but not necessarily, an ultraviolet light source or a corona discharging means or a combination of these.

The air outlet 10 of the housing 8 is functionally connected to a thermally insulated interior 4 for storing ice (not shown in the figures) of the ice bin 39 for feeding air containing ozone from the housing 8 of the ozone generator 7 to the thermally insulated interior 4 of the ice bin 39, and the air inlet 9 of the housing 8 of the ozone generator 7 being functionally connected to the thermally insulated interior 4 of the ice bin 39 for receiving air from the thermally insulated interior 4 of the ice bin 39. In FIGS. 1 to 4, the ice bin 39 is provided with a closable and openable hinged lid 40 for accessing the thermally insulated interior 4 from the outside of the ice bin 39.

The air inlet 9 of the housing 8 of the ozone generator 7 is preferably, but not necessarily, functionally connected with the thermally insulated interior 4 of the ice bin 39 to receive air solely from the thermally insulated interior 4 of the ice bin 39.

In the ice bin 39 shown in FIG. 2, the ozone generator 7 is placed outside the thermally insulated interior 4 of the ice bin 39.

In FIG. 2, the ice bin 39 is provided with two apertures 32 provided between the outside of the thermally insulated interior 4 of the ice bin 39 and the thermally insulated interior 4 of the ice bin 39. The air outlet 10 of the housing 8 of the ozone generator 7 is functionally connected with an aperture 32 of the ice maker 1 for feeding air containing ozone from the ozone generator 7 into the thermally insulated interior 4 of the ice bin 39 and the air inlet 9 of the housing 8 of the ozone generator 7 is functionally connected with an aperture 32 of the ice maker 1 for receiving air from the thermally insulated interior 4 of the ice bin 39 with the ozone generator 7.

In FIG. 2, the air inlet 9 of the ozone generator 7 comprises a first tube 30 for leading air into the housing 8 of the ozone generator 7 and the air outlet 10 of the ozone generator 7 comprises a second tube 31 for feeding air from the housing 8 of the ozone generator 7. The first tube 30 is fitted in an aperture 32 of the ice bin 39 and the second tube 31 is fitted in an aperture 32 of the ice bin 39.

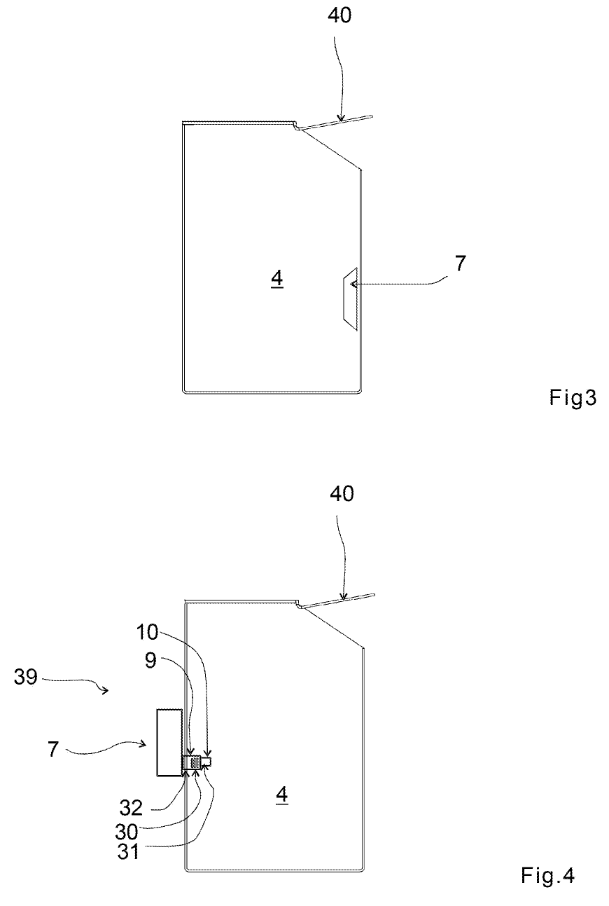

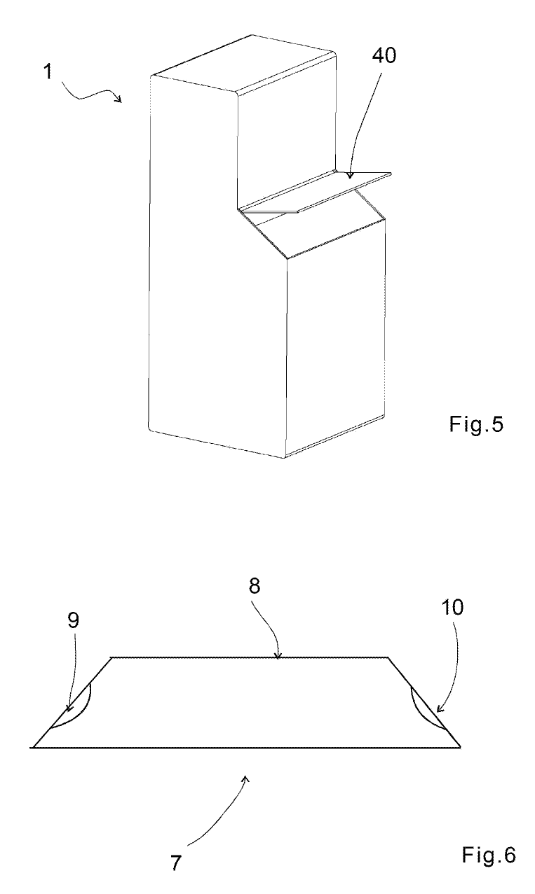

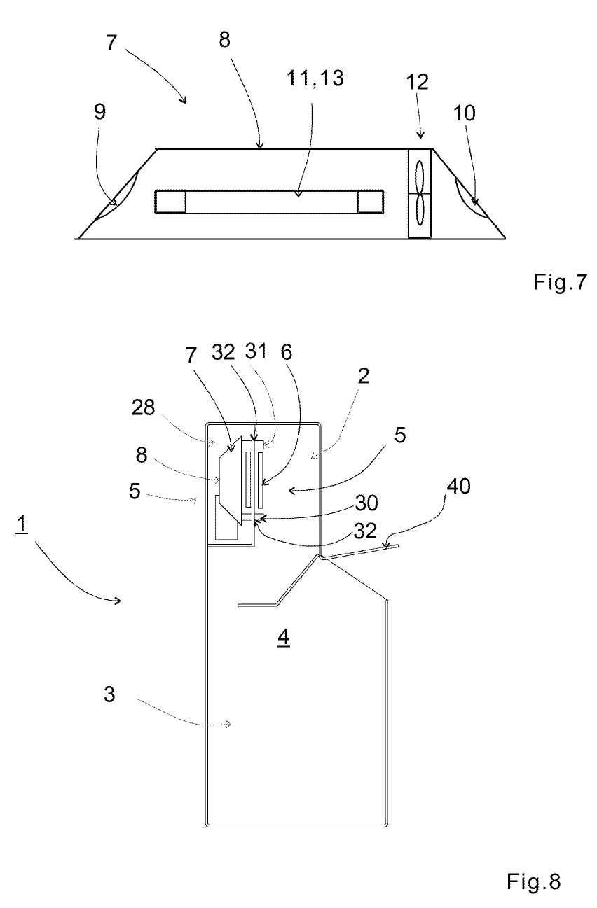

In the ice bin 39 shown in FIG. 3, the ozone generator 7 is placed in the thermally insulated interior 4 of the ice bin 39.

In FIG. 4 the ozone generator 7 is placed outside of the thermally insulated interior 4 of the ice bin 39. The ice bin 39 comprises a single aperture 32 between the outside of the thermally insulated interior 4 and the inside of the thermally insulated interior 4 for both the air inlet 9 of the ozone generator 7 to be functionally connected with the thermally insulated interior 4 of the ice bin 39 and the air outlet 10 of the ozone generator 7 to be functionally connected with the thermally insulated interior 4 of the ice bin 39.

In FIG. 4, the air outlet 10 of the ozone generator 7 comprises a second tube 31, which is partly placed inside a first tube 30 of the air inlet 9 of the ozone generator 7 to firstly allow the placement of the ozone generator 7 outside the thermally insulated interior 4 of the ice bin 39 and to secondly allow both the air outlet 10 of the ozone generator 7 and the air inlet 9 of the ozone generator 7 to be in communication with the thermally insulated interior 4 of the ice bin 39 via the single aperture 32 between the outside of the thermally insulated interior 4 and the inside of the thermally insulated interior 4.

FIG. 19 shows in cross-section view an ozone generator 7, which can be used in an ice bin 39 of the embodiment shown in FIG. 4. In a ozone generator shown in FIG. 19, air is drawn from the thermally insulated interior 4 into the first tube 30 connected to the air inlet 9 via the holes 41 provided in the first tube 30 by means of a pump 12 arranged in the housing 8 of the ozone generator 7. The purpose of the pump 12 is to create a flow of air through the housing 8. After the first tube 30 the flow of air enters the housing 8 of the ozone generator 7, in which ozone generating means 11 are arranged for generating ozone in the air flowing through the housing 8. After this, air containing ozone flows from the housing 8 into the second tube 31 of the air outlet 10 and air containing ozone flows through the second tube 31 and into the thermally insulated interior 4.

The invention also relates to a method for cleaning an ice bin 39 for storing ice.

The method comprises a step of providing an ozone generator 7 for generating ozone, the ozone generator 7 comprising a housing 8 having an air inlet 9 and an air outlet 10, wherein the air inlet 9 of the housing 8 of the ozone generator 7 is functionally connected to the air outlet 10 of the housing 8 of the ozone generator 7 for allowing air to flow through the housing 8, and ozone generating means 11 in the housing 8 for generating ozone in the air flowing through the housing 8. The ozone generating means 11 is preferably, but not necessarily, an ultraviolet light source or a corona discharging means or a combination of these.

The method comprises the steps of coupling the air inlet 9 of the housing 8 of the ozone generator 7 to n thermally insulated interior 4 for storing ice of the ice bin 39 and coupling the air outlet 10 of the housing 8 to an thermally insulated interior 4 of the ice maker 1.

The method comprises the steps of operating the ozone generator 7 to cause air to flow from the thermally insulated interior 4 of the ice bin 39 through the housing 8 of the ozone generator 7 and back to the thermally insulated interior 4 of the ice bin 39 and operating the ozone generating means 11 inside the housing 8 for generating ozone in the air flowing through the housing 8.

In a preferred embodiment of the method for cleaning an ice bin 39, the method comprises the steps of providing at least one aperture 32 in the ice bin 39 between the outside of the thermally insulated interior 4 of the ice bin 39 and the inside of the thermally insulated interior 4 of the ice bin 39, and arranging the ozone generator 7 outside the thermally insulated interior 4 of the ice bin 39, and coupling the air inlet 9 of the ozone generator 7 and the air outlet 10 of the ozone generator 7 with the at least one aperture 32 in the ice bin 39 between the outside of the thermally insulated interior 4 of the ice bin 39 and the inside of the thermally insulated interior 4 of the ice bin 39.

In a preferred embodiment of the method for cleaning an ice bin 39, the method comprises the steps of providing at least one aperture 32 in the ice bin 39 between the outside of the thermally insulated interior 4 of the ice bin 39 and the thermally insulated interior 4 of the ice bin 39, providing the air inlet 9 of the ozone generator 7 with a first tube 30 for leading air into the housing 8 of the ozone generator 7, providing the air outlet 10 of the ozone generator 7 with a second tube 31 for feeding air from the housing 8 of the ozone generator 7, and arranging the first tube 30 and the second tube 31 in the at least one aperture 32 so that the ozone generator 7 is placed outside the thermally insulated interior 4 of the ice bin 39 and the first tube 30 and the second tube 31 are functionally connected with the thermally insulated interior 4 of the ice maker 1.

In a preferred embodiment of the method for cleaning an ice bin 39, the method comprises a step of providing one aperture 32 in the ice bin 39 between the outside of the thermally insulated interior 4 of the ice bin 39 and the thermally insulated interior 4 of the ice bin 39. This preferred embodiment of the method includes the steps of providing the air inlet 9 of the ozone generator 7 with a first tube 30 for leading air into the housing 8 of the ozone generator 7 and providing the air outlet 10 of the ozone generator 7 with a second tube 31 for feeding air from the housing 8 of the ozone generator 7. This preferred embodiment of the method includes the step of arranging both the first tube 30 and the second tube 31 in the one aperture 32 so that the ozone generator 7 is placed outside the thermally insulated interior 4 of the ice bin 39 and the first tube 30 and the second tube 31 are functionally connected with the thermally insulated interior 4 of the ice bin 39. This preferred embodiment of the method includes preferable a step of arranging the first tube 30 inside the second tube 31 or arranging the second tube 31 inside the first tube 30 (as shown in FIG. 4) in the one aperture 32 so that the ozone generator 7 is placed outside the thermally insulated interior 4 of the ice bin 39 and the first tube 30 and the second tube 31 are functionally connected with the thermally insulated interior 4 of the ice bin 39.

The invention also relates to an ice maker for making ice.

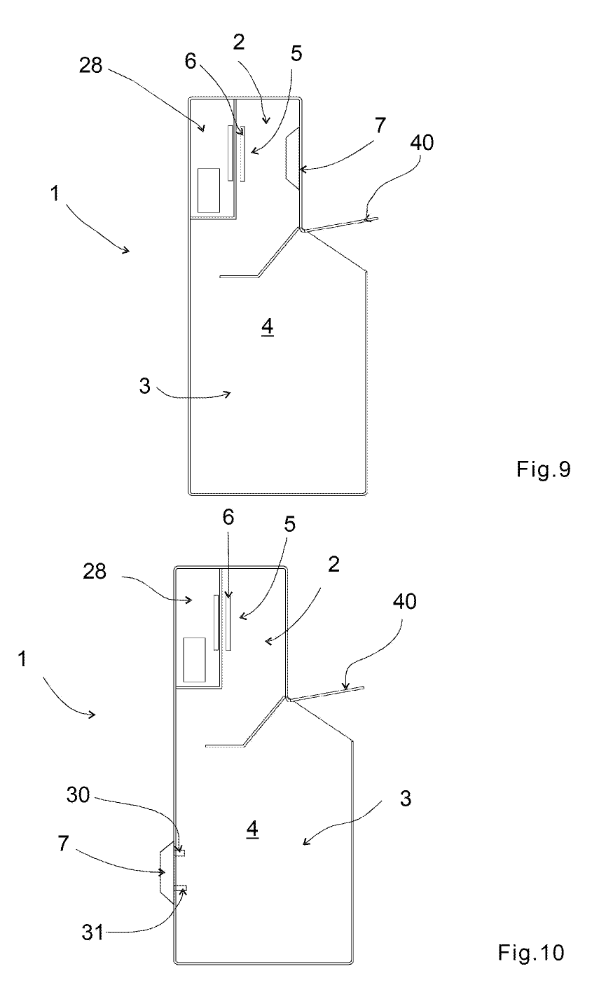

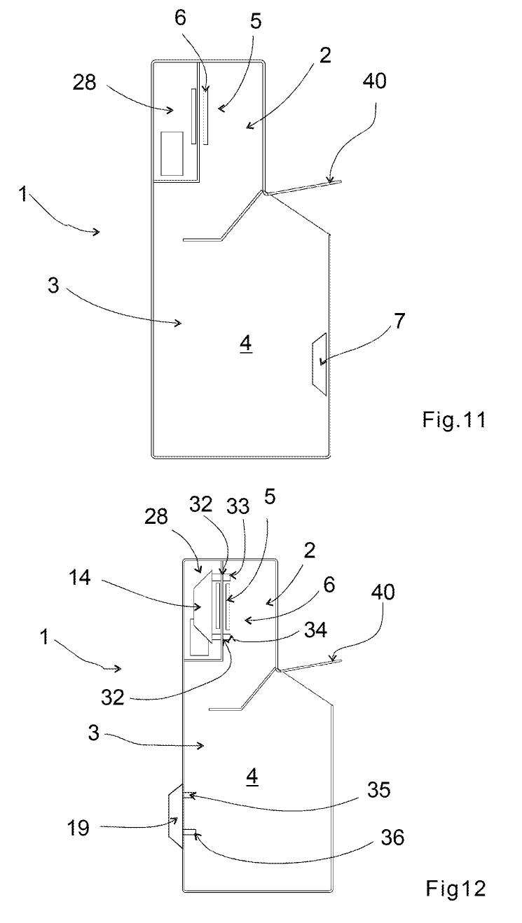

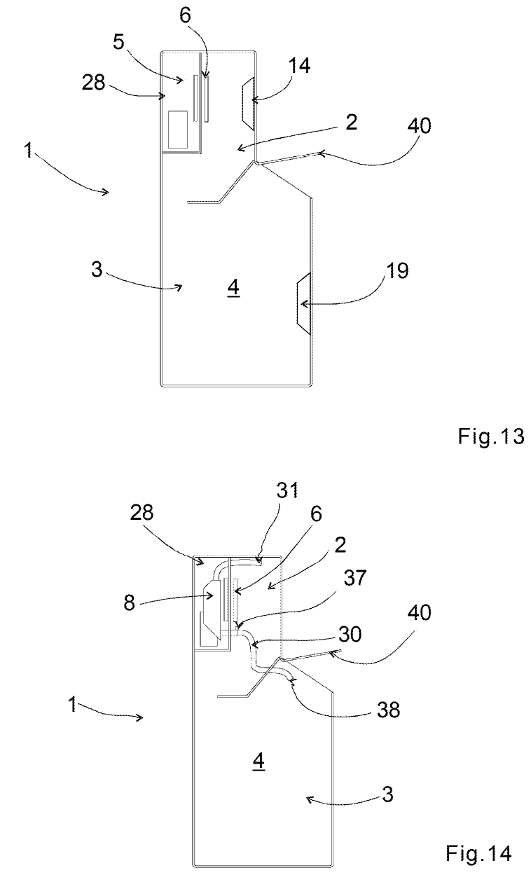

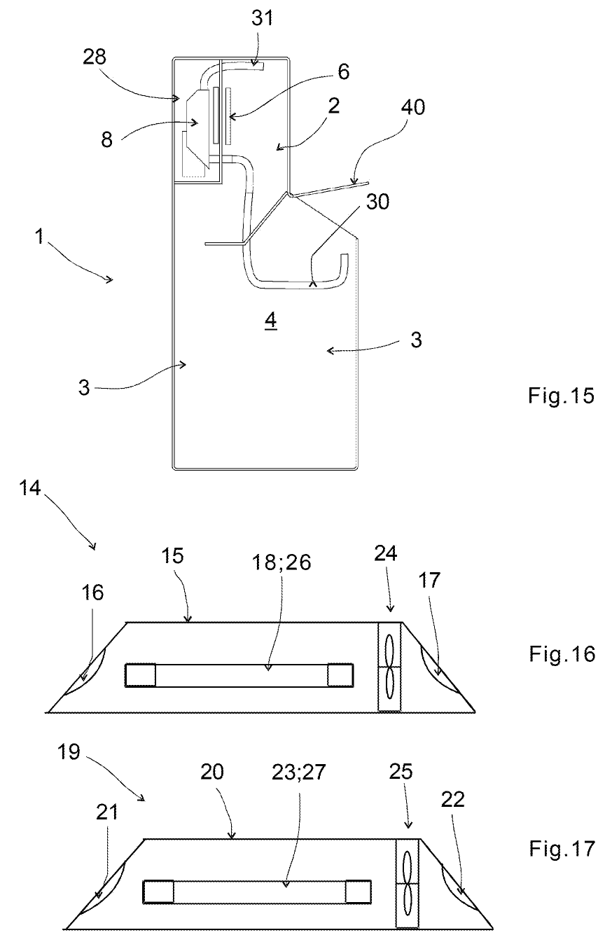

FIGS. 8 to 15 show an ice maker. The ice maker 1 shown in FIGS. 8 to 15 comprises an ice making section 2, a refrigeration section 28 functionally connected with the ice making section 2, and an ice storage section 3 for storing ice (not shown in the figures) produced in the ice making section 2.

The ice maker 1 shown in FIGS. 8 to 15 comprises n thermally insulated interior 4, which is divided into the ice making section 2 and the ice storage section 3. In FIGS. 8 to 15, the ice maker 1 is provided with a closable and openable hinged lid 40 for accessing the thermally insulated interior 4 from the outside of the ice maker 1.

A refrigeration section 28 of a ice maker comprises ice making means 5 typically including a compressor (not shown in the figures), a condenser (not shown in the figures), condenser pump (not shown in the figures) and associated electronics and other devices (not shown in the figures) for the operation thereof. An evaporator mold 6 of the ice making means 5 for forming ice such as ice cubes is located in the ice making section 2. Water distributing means (not shown in the figures) for distributing water into the evaporator mold 6 is also arranged in the ice making section 2. The basic function of an ice maker 1 is well known in the art. Basically and shortly water is distributed by the water distributing means into the evaporator mold, which is cooled below the freezing point of water by means of the compressor, the condenser and the condenser pump to make ice of the water in the evaporator mold. The ice or ice cubes produced in the ice making section 2 are thereafter released from the evaporator mold 6 and dropped into the ice storage section.

The ice maker 1 comprises an ozone generator 7 for generating ozone (not shown in the figures).

The ozone generator 7 comprises a housing 8 having an air inlet 9 and an air outlet 10 for allowing air to flow through the housing 8. The air inlet 9 of the housing 8 of the ozone generator 7 is functionally connected to the air outlet 10 of the housing 8 of the ozone generator 7 for allowing air to flow through the housing 8.

The ozone generator 7 comprises ozone generating means 11 in the housing 8 or functionally connected with the housing 8 for generating ozone in air flowing through the housing 8.

The air outlet 10 of the housing 8 is functionally connected to an thermally insulated interior 4 of the ice maker 1 to discharge air containing ozone from the housing 8 of the ozone generator 7 to the thermally insulated interior 4 of the ice maker 1.

The air inlet 9 of the housing 8 of the ozone generator 7 is functionally connected to the thermally insulated interior 4 of the ice maker 1 to receive air from the thermally insulated interior 4 of the ice maker 1.

The air inlet 9 of the housing 8 of the ozone generator 7 is preferably, but not necessarily, functionally connected with the thermally insulated interior 4 of the ice maker 1 to receive air solely from the thermally insulated interior 4 of the ice maker 1.

The ozone generator 7 comprises preferably, but not necessarily, a pump 12 in the housing 8 for generating the airflow through the housing 8.

The ozone generating means 11 in the housing 8 of the ozone generator 7 comprises preferably, but not necessarily, an ultraviolet light source 13 for generating ozone in the air flowing through the housing 8. Alternatively the ozone generating means 11 in the housing 8 of the ozone generator 7 may comprise corona discharge means (not shown in the figures) for generating ozone in the air flowing through the housing 8.

The ozone generator 7 is preferably, but not necessarily, arranged in the thermally insulated interior 4 of the ice maker 1. Alternatively the ozone generator 7 may be arranged in the refrigeration section 28 of the ice maker 1.

The ozone generator 7 can alternatively be placed outside the thermally insulated interior 4 of the ice maker 1. In this embodiment the ice maker 1 is provided with at least one aperture 32 provided between the outside of the ice making section 2 of the thermally insulated interior 4 of the ice maker 1 and the ice making section 2 of the thermally insulated interior 4 of the ice maker 1. In this embodiment at least one of the air outlet 10 of the ozone generator 7 and the air inlet 9 of the ozone generator 7 are functionally connected with the at least one aperture 32 provided between the outside of the ice making section 2 of the thermally insulated interior 4 of the ice maker 1 and the ice making section 2 of the thermally insulated interior 4 of the ice maker 1 for feeding air containing ozone from the ozone generator 7 into the ice making section 2 of the thermally insulated interior 4 of the ice maker 1, or respectively, for receiving air from the ice making section 2 of the thermally insulated interior 4 of the ice maker 1 with the ozone generator 7.

In FIGS. 8 and 10 the air inlet 9 of the ozone generator 7 comprises a first tube 30 for leading air into the housing 8 of the ozone generator 7 and air outlet 10 of the ozone generator 7 comprises a second tube 31 for feeding air from the housing 8 of the ozone generator 7. The first tube 30 may be fitted in an aperture 32 and the second tube 31 may be fitted in an aperture 32 of the ice maker 1 so that the ozone generator 7 is placed outside the thermally insulated interior 4 of the ice maker 1 and the first tube 30 and the second tube 31 are functionally connected with the thermally insulated interior 4 of the ice maker 1. The first tube 30 and the second tube 31 are preferably, but not necessarily, arranged at least partly co-axially so that the first tube 30 and the second tube 31 are both fitted in one and the same aperture 32 of the ice maker 1.

The thermally insulated interior 4 of the ice maker 1 comprises in a preferred embodiment an ice making section 2 comprising ice making means 5 for making ice and an ice storage section 3 for storing ice, the ice storage section 3 being configured to receive ice from an ice making section 2 of the thermally insulated interior 4 of the ice maker 1. In this preferred embodiment is at least one of the air inlet 9 and the air outlet 10 of the housing 8 of the zone generator is functionally connected with the ice making section 2. In this preferred embodiment is preferably both the air inlet 9 and the air outlet 10 of the housing 8 of the zone generator functionally connected with the ice making section 2. The air inlet 9 of the housing 8 of the ozone generator 7 is preferably, but not necessarily, functionally connected with the ice making section 2 of the thermally insulated interior 4 of the ice maker 1 to receive air solely from the ice making section 2 of the thermally insulated interior 4 of the ice maker 1.

In a preferred embodiment of the invention the ozone generator 7 is placed outside the thermally insulated interior 4 of the ice maker 1. In this preferred embodiment the ice maker 1 is provided with at least one aperture 32 provided between the outside of the ice making section 2 of the interior 4 of the ice maker 1 and the ice making section 2 of the interior 4 of the ice maker 1. In this preferred embodiment the air outlet 10 of the housing 8 of the ozone generator 7 and the air inlet 9 of the housing 8 of the ozone generator 7 are functionally connected with the at least one aperture 32 provided between the outside of the ice making section 2 of the thermally insulated interior 4 of the ice maker 1 and the ice making section 2 of the thermally insulated interior 4 of the ice maker 1 for feeding air containing ozone from the ozone generator 7 into the ice making section 2 of the thermally insulated interior 4 of the ice maker 1 and for receiving air from the ice making section 2 of the thermally insulated interior 4 of the ice maker 1 with the ozone generator 7.

In FIG. 8 the air inlet 9 of the ozone generator 7 comprises a first tube 30 for leading air into the housing 8 of the ozone generator 7 and the air outlet 10 of the ozone generator 7 comprises a second tube 31 for feeding air from the housing 8 of the ozone generator 7. The first tube 30 may be fitted in an aperture 32 and the second tube 31 may be fitted in an aperture 32 of the ice maker 1 so that the ozone generator 7 is placed outside the ice making section 2 of the thermally insulated interior 4 of the ice maker 1 and the first tube 30 and the second tube 31 are functionally connected with the ice making section 2 of the thermally insulated interior 4 of the ice maker 1. The first tube 30 and the second tube 31 are preferably, but not necessarily, arranged at least partly co-axially so that the first tube 30 and the second tube 31 are both fitted in one and the same aperture 32 of the ice maker 1.

The thermally insulated interior 4 of the ice maker 1 comprises in another preferred embodiment an ice making section 2 comprising ice making means 5 for making ice and an ice storage section 3 for storing ice, the ice storage section 3 being configured to receive ice from an ice making section 2 of the thermally insulated interior 4 of the ice maker 1. In this preferred embodiment is at least one of the air inlet 9 and the air outlet 10 of the housing 8 of the zone generator is functionally connected with the ice storage section. In this preferred embodiment is preferably both the air inlet 9 and the air outlet 10 is functionally connected with the ice storage section. The air inlet 9 of the housing 8 of the ozone generator 7 is preferably, but not necessarily, functionally connected with the ice storage section 3 of the thermally insulated interior 4 of the ice maker 1 to receive air solely from the ice storage section 3 of the thermally insulated interior 4 of the ice maker 1.

In a preferred embodiment the ozone generator 7 is placed outside the thermally insulated interior 4 of the ice maker 1. In this preferred embodiment the ice maker 1 is provided with at least one aperture 32 provided between the outside of the ice storage section 3 of the thermally insulated interior 4 of the ice maker 1 and the ice storage section 3 of the thermally insulated interior 4 of the ice maker 1. In a preferred embodiment the air outlet 10 of the ozone generator 7 and the air inlet 9 of the ozone generator 7 are functionally connected with the at least one aperture 32 provided between the outside of the ice storage section 3 of the thermally insulated interior 4 of the ice maker 1 and the ice storage section 3 of the thermally insulated interior 4 of the ice maker 1 for feeding air containing ozone from the ozone generator 7 into the ice storage section 3 of the thermally insulated interior 4 of the ice maker 1 and for receiving air from the ice storage section 3 of the thermally insulated interior 4 of the ice maker 1 with the ozone generator 7.

In FIG. 10 the air inlet 9 of the ozone generator 7 comprises a first tube 30 for leading air into the housing 8 of the ozone generator 7 and wherein the air outlet 10 of the ozone generator 7 comprises a second tube 31 for feeding air from the housing 8 of the ozone generator 7. The first tube 30 may be fitted in an aperture 32 and the second tube 31 may be fitted in an aperture 32 of the ice maker 1 so that the ozone generator 7 is placed outside the ice storage section 3 of the thermally insulated interior 4 of the ice maker 1 and the first tube 30 and the second tube 31 are functionally connected with the ice storage section 3 of the thermally insulated interior 4 of the ice maker 1. The first tube 30 and the second tube 31 are preferably, but not necessarily, arranged at least partly co-axially so that the first tube 30 and the second tube 31 are both fitted in one and the same aperture 32 of the ice maker 1.

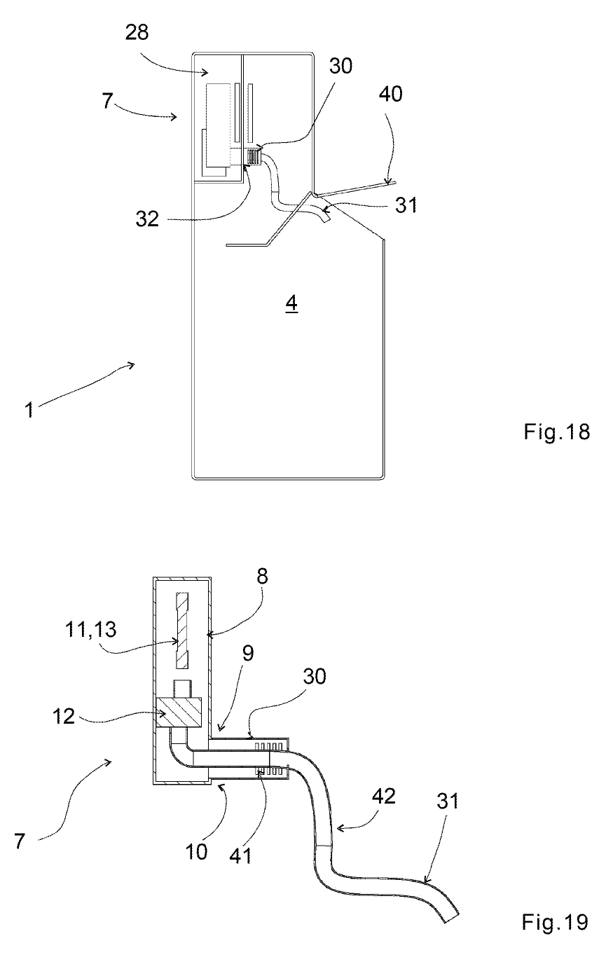

In the embodiment shown in FIG. 18, the ice maker 1 comprises a single aperture 32 between the outside of the thermally insulated interior 4 and the inside of the thermally insulated interior 4 for both the air inlet 9 of the ozone generator 7 and the air outlet 10 of the ozone generator 7.

In FIG. 18 the ozone generator 7 is placed in the refrigeration section 28 of the ice maker 1. In FIG. 18, the air outlet 10 of the ozone generator 7 comprises a second tube 31, which is partly placed inside a first tube 30 of the air inlet 9 of the ozone generator 7 to firstly allow the placement of the ozone generator 7 outside the thermally insulated interior 4 and to secondly allow both the air outlet 10 of the ozone generator 7 and the air inlet 9 of the ozone generator 7 to be in communication with the thermally insulated interior 4 via one single aperture 32 between the outside of the thermally insulated interior 4 and the inside of the thermally insulated interior 4.

FIG. 19 shows in greater detail and in cross-section the ozone generator 7 of the ice maker 1 shown in FIG. 18. In FIG. 19, the first tube 30 is provided with holes 41 to allow air from the thermally insulated interior 4 to enter the first tube 30. The second tube 31 may be perforated or provided with small holes 42 to allow air containing ozone to exit the second tube 31 before the air containing ozone arrives the end of the second tube 31. In a ozone generator shown in FIG. 19, air is drawn from the thermally insulated interior 4 into the first tube 30 connected to the air inlet 9 via the holes 41 provided in the first tube 30 by means of a pump 12 arranged in the housing 8 of the ozone generator 7. The purpose of the pump 12 is to create a flow of air through the housing 8. After the first tube 30 the flow of air enters the housing 8 of the ozone generator 7, in which ozone generating means 11 are arranged for generating ozone in the air flowing through the housing 8. After this, air containing ozone flows from the housing 8 into the second tube 31 of the air outlet 10 and air containing ozone flows through the second tube 31 and into the thermally insulated interior 4.

The thermally insulated interior 4 of the ice maker 1 comprises in another preferred embodiment an ice making section 2 comprising ice making means 5 for making ice and an ice storage section 3 for storing ice, the ice storage section 3 being configured to receive ice from an ice making section 2 of the thermally insulated interior 4 of the ice maker 1. In this preferred embodiment the ozone generator is a first ozone generator 14. In this preferred embodiment the first ozone generator 14 comprises a first housing 15 having a first air inlet 16 and a first air outlet 17. The first air inlet 16 of the first housing 15 of the first ozone generator 14 is functionally connected to the first air outlet 17 of the first housing 15 of the first ozone generator 14 for allowing air to flow through the first housing 15. In this preferred embodiment the first ozone generator 14 comprises first ozone generating means 18 in the first housing 15 or functionally connected to the first housing 15 for generating ozone in air flowing through the first housing 15. In this preferred embodiment the first air outlet 17 of the first housing 15 is functionally connected to the ice making section 2 of an thermally insulated interior 4 of the ice maker 1 to discharge air containing ozone from the first housing 15 of the first ozone generator 14 to the ice making section 2 of the thermally insulated interior 4 of the ice maker 1 and the first air inlet 16 of the first housing 15 of the first ozone generator 14 is functionally connected to the ice making section 2 of the thermally insulated interior 4 of the ice maker 1 to receive air from the ice making section 2 of the interior 4 of the ice maker 1.

In this preferred embodiment the ice maker 1 further comprises a second ozone generator 19. The second ozone generator 19 comprises a second housing 20 having a second air inlet 21 and a second air outlet 22. The second air inlet 21 of the second housing 20 of the second ozone generator 19 is functionally connected to the second air outlet 22 of the second housing 20 of the second ozone generator 19 for allowing air to flow through the second housing 20. The second ozone generator 19 comprises second ozone generating means 23 in the second housing 20 or functionally connected to the second housing 20 for generating ozone in air flowing through the second housing 20. The second air outlet 22 of the second housing 20 is functionally connected to the ice making section 2 of the thermally insulated interior 4 of the ice maker 1 to discharge air containing ozone from the second housing 20 of the second ozone generator 19 to the ice making section 2 of the thermally insulated interior 4 of the ice maker 1. The second inlet of the second housing 20 of the second ozone generator 19 is functionally connected to the ice making section 2 of the thermally insulated interior 4 of the ice maker 1 to receive air from the ice making section 2 of the thermally insulated interior 4 of the ice maker 1.

The first air inlet 16 of the first housing 15 of the first ozone generator 14 is preferably, but not necessarily, functionally connected to the ice making section 2 of the thermally insulated interior 4 of the ice maker 1 to receive air solely from the ice making section 2 of the thermally insulated interior 4 of the ice maker 1. The second air inlet 21 of the second housing 20 of the second ozone generator 19 is preferably, but not necessarily, functionally connected to the ice storage section 3 of the thermally insulated interior 4 of the ice maker 1 to receive air solely from the ice storage section 3 of the thermally insulated interior 4 of the ice maker 1.

In this preferred embodiment the first ozone generator 14 comprises preferably, but not necessarily, a first pump 24 in the first housing 15 for generating an airflow through the first housing 15, and the second ozone generator 19 comprises preferably, but not necessarily, a second pump 25 in the second housing 20 for generating an airflow through the second housing 20. In this preferred embodiment the first ozone generating means 18 in the first housing 15 of the first ozone generator 14 comprises preferably, but not necessarily, a first ultraviolet light source 26 for generating ozone in the first airflow, and the second ozone generating means 23 in the second housing 20 of the second ozone generator 19 comprises preferably, but not necessarily, a second ultraviolet light source 27 for generating ozone in the second airflow.

In a preferred embodiment the first ozone generator 14 and the second ozone generator 19 are placed outside the thermally insulated interior 4 of the ice maker 1. In this preferred embodiment the ice maker 1 is provided with at least one aperture 32 provided between the outside of the ice making section 2 of the thermally insulated interior 4 of the ice maker 1 and the ice making section 2 of the thermally insulated interior 4 of the ice maker 1 and the first air outlet 17 of the first ozone generator 14 and the first air inlet 16 of the first ozone generator 14 are functionally connected with the at least one aperture 32 provided between the outside of the ice making section 2 of the thermally insulated interior 4 of the ice maker 1 and the ice making section 2 of the thermally insulated interior 4 of the ice maker 1 for feeding air containing ozone from the first ozone generator 14 into the ice making section 2 of the thermally insulated interior 4 of the ice maker 1 and for receiving air from the ice making section 2 of the thermally insulated interior 4 of the ice maker 1 with the first ozone generator 14. In this preferred embodiment the ice maker 1 is provided with at least one aperture 32 provided between the outside of the ice storage section 3 of the thermally insulated interior 4 of the ice maker 1 and the ice storage section 3 of the thermally insulated interior 4 of the ice maker 1 and the second air outlet 22 of the second ozone generator 19 and the second air inlet 21 of the second ozone generator 19 are functionally connected with the at least one aperture 32 provided between the outside of the ice storage section 3 of the thermally insulated interior 4 of the ice maker 1 and the ice storage section 3 of the thermally insulated interior 4 of the ice maker 1 for feeding air containing ozone from the second ozone generator 19 into the ice storage section 3 of the thermally insulated interior 4 of the ice maker 1 and for receiving air from the ice storage section 3 of the thermally insulated interior 4 of the ice maker 1 with the second ozone generator 19.

In FIG. 12 the first air inlet 17 of the first ozone generator 14 comprises a third tube 33 for leading air into the first housing 15 of the first ozone generator 17 and the first air outlet 17 of the first ozone generator 14 comprises a fourth tube 34 for feeding air from the first housing 15 of the first ozone generator 14.

In FIG. 12 the third tube 33 is fitted in an aperture 32 and the fourth tube 34 is fitted in an aperture 32 of the ice maker 1 so that the first ozone generator 14 is placed outside the ice making section 2 of the thermally insulated interior 4 of the ice maker 1 and the third tube 33 and the fourth are functionally connected with the ice making section 2 the thermally insulated interior 4 of the ice maker 1. The third tube 33 and the fourth tube 34 may be arranged at least partly co-axially so that the third tube 33 and the fourth tube 34 are fitted in one and the same aperture.

In FIG. 12 the second air inlet 21 of the second ozone generator 19 comprises a fifth tube 35 for leading air into the second housing 20 of the second ozone generator 19 and the second air outlet 22 of the second ozone generator 19 comprises a sixth tube 36 for feeding air from the second housing 8 of the second ozone generator 19.

In FIG. 12 the fifth tube 35 is fitted in an aperture 32 and the sixth tube 36 fitted in an aperture 32 of the ice maker 1 so that the ozone generator 7 is placed outside the ice storage section 3 of the thermally insulated interior 4 of the ice maker 1 and the fifth tube 35 and the sixth tube 36 are functionally connected with the ice storage section 3 of the thermally insulated interior 4 of the ice maker 1. The fifth tube 35 and the sixth tube 36 may be arranged at least partly co-axially so that the fifth tube 35 and the sixth tube 36 are fitted in one and the same aperture.

In the ice maker shown in FIG. 14, a first tube 30 of the ozone generator 7 is arranged to lead air from an ice making section 1 of the ice maker 1 to the ozone generator 7 and a second tube 31 of the ozone generator 7 is provided with a first outlet 37 and a second outlet 38. The first outlet 37 is arranged in an ice making section 2 of the ice maker 1 to feed air containing ozone into the ice making section 2 of the ice maker 1 and the second outlet 38 is arranged in an ice storage section 3 of the ice maker 1 to feed air containing ozone into the ice storage section 3 of the ice maker 1.

In the ice maker shown in FIG. 14, a first tube 30 of the ozone generator 7 is arranged to lead air from an ice making section 1 of the ice maker 1 to the ozone generator 7 and a second tube 31 of the ozone generator 7 is arranged to feed air containing ozone in an ice storage section 3 of the ice maker 1.

To feed air contain ozone to an ice storage section 3 of an ice maker 1 is especially advantageous, because an ice storage section 3 of an ice maker 1 is a space of an ice maker 1 is provided with an lid or door for accessing the ice in the ice storage section 3. When the lid or door is opened and ice is taken from the ice storage section 3 with for example a scoop (not shown in the figures), contaminates from the scoop and the surrounding air can freely access the ice in the ice storage section 3 and contaminate the ice.

The invention also relates to a method for cleaning an ice maker 1 for making ice. The method comprises a step of providing an ozone generator 7 for generating ozone. The ozone generator 7 comprises a housing 8 having an air inlet 9 and an air outlet 10, wherein the air inlet 9 of the housing 8 of the ozone generator 7 is functionally connected to the air outlet 10 of the housing 8 of the ozone generator 7 for allowing air to flow through the housing 8, and ozone generating means 11 in the housing 8 for generating ozone in the air flowing through the housing 8.

The method comprises in addition a step of coupling the air inlet 9 of the housing 8 of the ozone generator 7 to receive air from a thermally insulated interior 4 of the ice maker 1.

The method comprises in addition a step of coupling the air outlet 10 of the housing 8 to an thermally insulated interior 4 of the ice maker 1 to receive air containing ozone from the thermally insulated interior 4 of the ozone generator 7.

The method comprises in addition a step of operating the ozone generator 7 to cause air to flow from the interior 4 of the ice maker 1 through the housing 8 of the ozone generator 7 and back to the thermally insulated interior 4 of the ice maker 1.

The method comprises in addition a step of operating the ozone generating means 11 inside the housing 8 for generating ozone in the air flowing through the housing 8.

The method comprises preferably a step of coupling the air inlet 9 of the housing 8 of the ozone generator 7 to receive air solely from the thermally insulated interior 4 of the ice maker 1.

In a preferred embodiment of the method, the method including the step of arranging the ozone generator 7 in the thermally insulated interior 4 of the ice maker 1.

In a preferred embodiment of the method, the method including the step of providing at least one aperture 32 in the ice maker 1 between the outside of the ice making section 2 of the thermally insulated interior 4 of the ice maker 1 and the ice making section 2 of the thermally insulated interior 4 of the ice maker 1. This preferred embodiment includes the steps of arranging the ozone generator 7 outside the thermally insulated interior 4 of the ice maker 1 and coupling the air inlet 9 of the ozone generator 7 and the air outlet 10 of the ozone generator 7 with the at least one aperture 32 in the ice maker 1 between the outside of the ice making section 2 of the thermally insulated interior 4 of the ice maker 1 and the ice making section 2 of the thermally insulated interior 4 of the ice maker 1.

In a preferred embodiment of the method, the method including the steps of providing the air inlet 9 of the ozone generator 7 with a first tube 30 for leading air into the housing 8 of the ozone generator 7 and providing the air outlet 10 of the ozone generator 7 with a second tube 31 for feeding air from the housing 8 of the ozone generator 7 and arranging the first tube 30 and the second tube 31 in an aperture 32 of the ice maker 1 so that the ozone generator 7 is placed outside the thermally insulated interior 4 of the ice maker 1 and the first tube 30 and the second tube 31 are functionally connected with the thermally insulated interior 4 of the ice maker 1.

In a preferred embodiment of the method, the method including the steps of providing the air inlet 9 of the ozone generator 7 with a first tube 30 for leading air into the housing 8 of the ozone generator 7 and providing the air outlet 10 of the ozone generator 7 with a second tube 31, at least partly co-axial with the first tube 30, for feeding air from the housing 8 of the ozone generator 7 and arranging the first tube 30 and the second tube 31 in an aperture 32 of the ice maker 1 so that the ozone generator 7 is placed outside the thermally insulated interior 4 of the ice maker 1 and the first tube 30 and the second tube 31 are functionally connected with the thermally insulated interior 4 of the ice maker 1.

In a preferred embodiment of the method for cleaning an ice maker 1, the method comprises a step of providing one aperture 32 in the ice maker 1 between the outside of the thermally insulated interior 4 of the ice maker 1 and the inside of the thermally insulated interior 4 of the ice maker 1. This preferred embodiment of the method includes the steps of providing the air inlet 9 of the ozone generator 7 with a first tube 30 for leading air into the housing 8 of the ozone generator 7 and providing the air outlet 10 of the ozone generator 7 with a second tube 31 for feeding air from the housing 8 of the ozone generator 7. This preferred embodiment of the method includes the step of arranging both the first tube 30 and the second tube 31 in the one aperture 32 so that the ozone generator 7 is placed outside the thermally insulated interior 4 of the ice maker 1 and the first tube 30 and the second tube 31 are functionally connected with the thermally insulated interior 4 of the ice maker 1. This preferred embodiment of the method includes preferable a step of arranging the first tube 30 inside the second tube 31 or arranging the second tube 31 inside the first tube 30 (as shown in FIG. 18) in the one aperture 32 so that the ozone generator 7 is placed outside the thermally insulated interior 4 of the ice maker 1 and the first tube 30 and the second tube 31 are functionally connected with the thermally insulated interior 4 of the ice maker 1.

In a preferred embodiment of the method, the method including a step of coupling the air inlet 9 of the ozone generator 7 to receive air from an ice making section 2 of the thermally insulated interior 4 of the ice maker 1. In this preferred embodiment of the method of the method of the invention the method comprises preferably, but not necessarily, a step of coupling the air inlet 9 of the housing 8 of the ozone generator 7 to receive air solely from the ice making section 2 of the thermally insulated interior 4 of the ice maker 1.

In a preferred embodiment of the method, the method including steps of arranging the ozone generator 7 in an ice making section 2 of the thermally insulated interior 4 of the ice maker 1 and coupling the air inlet 9 of the ozone generator 7 to receive air from the ice making section 2 of the thermally insulated interior 4 of the ice maker 1. In this preferred embodiment of the method of the method of the invention the method comprises preferably, but not necessarily, a step of coupling the air inlet 9 of the housing 8 of the ozone generator 7 to receive air solely from the ice making section 2 of the thermally insulated interior 4 of the ice maker 1.

In a preferred embodiment of the method, the method including steps of providing at least one aperture 32 in the ice maker 1 between the outside of the ice making section 2 of the thermally insulated interior 4 of the ice maker 1 and the ice making section 2 of the thermally insulated interior 4 of the ice maker 1 and arranging the ozone generator 7 outside the interior 4 of the ice maker 1. This preferred embodiment includes the step of coupling the air inlet 9 of the ozone generator 7 and the air outlet 10 of the ozone generator 7 with the at least one aperture 32 in the ice maker 1 between the outside of the ice making section 2 of the thermally insulated interior 4 of the ice maker 1 and the ice making section 2 of the thermally insulated interior 4 of the ice maker 1.

In a preferred embodiment of the method, the method including steps of providing the air inlet 9 of the ozone generator 7 with a first tube 30 for leading air into the housing 8 of the ozone generator 7 and providing the air outlet 10 of the ozone generator 7 with a second tube 31 for feeding air from the housing 8 of the ozone generator. This preferred embodiment includes the step of arranging the first tube 30 and the second tube 31 in at least one aperture 32 between the outside of the ice making section 2 of the thermally insulated interior 4 of the ice maker 1 and the ice making section 2 of the thermally insulated interior 4 of the ice maker 1 so that the ozone generator 7 is placed outside the ice making section 2 of the thermally insulated interior 4 of the ice maker 1 and the first tube 30 and the second tube 31 are functionally connected with the ice making section 2 of the thermally insulated interior 4 of the ice maker 1.

In a preferred embodiment of the method, the method including a step of coupling the air inlet 9 of the ozone generator 7 to receive air from an ice storage section 3 of the thermally insulated interior 4 of the ice maker 1, the ice storage section 3 being configured to receive ice from an ice making section 2 of the thermally insulated interior 4 of the ice maker 1. In this preferred embodiment of the method of the method of the invention the method comprises preferably, but not necessarily, a step of coupling the air inlet 9 of the housing 8 of the ozone generator 7 to receive air solely from the ice storage section 3 of the thermally insulated interior 4 of the ice maker 1.

In a preferred embodiment of the method, the method including steps of arranging the ozone generator 7 in an ice storage section 3 of the thermally insulated interior 4 of the ice maker 1, the ice storage section 3 being configured to receive ice from an ice making section 2 of the thermally insulated interior 4 of the ice maker 1, and coupling the air inlet 9 of the ozone generator 7 to receive air from the ice storage section 3 of the thermally insulated interior 4 of the ice maker 1. In this preferred embodiment of the method of the method of the invention the method comprises preferably, but not necessarily, a step of coupling the air inlet 9 of the housing 8 of the ozone generator 7 to receive air solely from the ice storage section 3 of the thermally insulated interior 4 of the ice maker 1.

In a preferred embodiment of the method, the method including the steps of providing at least one aperture 32 in the ice maker 1 between the outside of the ice storage section 3 of the thermally insulated interior 4 of the ice maker 1 and the ice storage section 3 of the thermally insulated interior 4 of the ice maker 1 and arranging the ozone generator 7 outside the thermally insulated interior 4 of the ice maker 1. This preferred embodiment of the method includes the step of coupling the air inlet 9 of the ozone generator 7 and the air outlet 10 of the ozone generator 7 with the at least one aperture 32 in the ice maker 1 between the outside of the ice storage section 3 of the thermally insulated interior 4 of the ice maker 1 and the ice storage section 3 of the thermally insulated interior 4 of the ice maker 1.

In a preferred embodiment of the method, the method including the steps of providing the air inlet 9 of the ozone generator 7 with a first tube 30 for leading air into the housing 8 of the ozone generator 7 and providing the air outlet 10 of the ozone generator 7 with a second tube 31 for feeding air from the housing 8 of the ozone generator 7, and arranging the first tube 30 and the second tube 31 in an aperture 32 of the ice maker 1 so that the ozone generator 7 is placed outside the thermally insulated interior 4 of the ice maker 1 and the first tube 30 and the second tube 31 are functionally connected with the ice storage section 3 of the thermally insulated interior 4 of the ice maker 1.

In a preferred embodiment of the method, the method including the steps of providing the air inlet 9 of the ozone generator 7 with a first tube 30 for leading air into the housing 8 of the ozone generator 7 and providing the air outlet 10 of the ozone generator 7 with a second tube 31, at least partly co-axial with the first tube 30, for feeding air from the housing 8 of the ozone generator 7, and arranging the first tube 30 and the second tube 31 in an aperture 32 between the outside of the ice storage section 3 of the thermally insulated interior 4 of the ice maker 1 and the ice storage section 3 of the thermally insulated interior 4 of the ice maker 1 so that the ozone generator 7 is placed outside the thermally insulated interior 4 of the ice maker 1 and the first tube 30 and the second tube 31 are functionally connected with the ice storage section 3 of the thermally insulated interior 4 of the ice maker 1.

In a preferred embodiment of the method, the method including the step of arranging ozone generating means 11 in the form of an ultraviolet light source 13 in the housing 8 for generating ozone in the air flowing through the housing 8.

In a preferred embodiment of the method, the method including the step of arranging ozone generating means 11 in the form of corona discharging means in the housing 8 for generating ozone in the air flowing through the housing 8.

In a preferred embodiment of the method, the method including steps of providing the ozone generator in the form of a first ozone generator 14, the first ozone generator 14 comprising a first housing 15 having a first air inlet 16 and a first air outlet 17, wherein the first air inlet 16 of the first housing 15 of the first ozone generator 14 is functionally connected to the first air outlet 17 of the first housing 15 of the first ozone generator 14 for allowing air to flow through the first housing 15, and first ozone generating means 18 in the first housing 15 for generating ozone in air flowing through the first housing 15.

This preferred embodiment includes the steps of coupling the first air inlet 16 of the first ozone generator 14 to receive air from the ice making section 2 of the thermally insulated interior 4 of the ice maker 1 and coupling the first air outlet 17 of the first housing 15 to discharge air from the first air outlet 17 of the first housing 15 into the ice making section 2 of the thermally insulated interior 4 of the ice maker 1. This preferred embodiment includes the steps of operating the first ozone generator 14 to cause air to flow from ice making section 2 of the interior 4 of the ice maker 1 through the housing of the ozone generator and back to the ice making section 2 interior 4 of the ice maker 1 and operating the first ozone generating means 18 inside the first housing 15 for generating ozone in air flowing through the first housing 15.

This preferred embodiment includes a step of providing in addition to the first ozone generator 14 a second ozone generator 19 for generating ozone, the second ozone generator 19 comprising a housing having a second air inlet 21 and a second air outlet 22, wherein the second air inlet 21 of the second housing 20 of the second ozone generator 19 is functionally connected to the second air outlet 22 of the second housing 20 of the second ozone generator 19 for allowing air to flow through the second housing 20, and second ozone generating means 23 in the second housing 20 for generating ozone in air flowing thorough the second housing 20. This preferred embodiment includes the steps of coupling the second air inlet 21 of the second housing 20 to receive air from an ice storage section 3 of the thermally insulated interior 4 of the ice maker 1 and coupling the second air outlet 22 of the second housing 20 to discharge air from the second air outlet 22 of the second housing 20 into the ice storage section 3 of the thermally insulated interior 4 of the ice maker 1. This preferred embodiment includes the steps of operating the second ozone generator 19 to cause air to flow from ice storage section 3 of the thermally insulated interior 4 of the ice maker 1 through the second housing 20 of the second ozone generator 19 and back to the ice making section 2 of the thermally insulated interior 4 of the ice maker 1 operating the second ozone generating means 23 inside the second housing 20 for generating ozone in air flowing through the second housing 20.

This preferred embodiment of the method includes preferably, but not necessary, steps of arranging the first ozone generator 14 in the ice making section 2 of the thermally insulated interior 4 of the ice maker 1 and arranging the second ozone generator 19 in the ice storage section 3 of the thermally insulated interior 4 of the ice maker 1. This preferred embodiment of the method includes preferably, but not necessarily, a step of arranging a first ozone generator 14 including a first pump 24 for generating a flow of air through the first housing 15 of the first ozone generator 14 and a step of arranging a second ozone generator 19 including a second pump 25 for generating a flow of air through the second housing 20 of the second ozone generator 19.

This preferred embodiment of the method includes preferably, but not necessary as step of arranging a first ozone generator 14 including first ozone generating means 18 in the form of a first ultraviolet light source 26 or first corona discharging means in the first housing 15 for generating ozone in the air flowing through the first housing 15. This preferred embodiment of the method includes preferably, but not necessary as step of arranging a second ozone generator 19 including second ozone generating means 23 in the form of a second ultraviolet light source 27 or second corona discharging means in the first housing 15 for generating ozone in the air flowing through the second housing 20. This preferred embodiment of the method includes preferably, but not necessarily, the steps of providing at least one aperture 32 in the ice maker 1 between the outside of the ice making section 2 of the thermally insulated interior 4 of the ice maker 1 and the ice making section 2 of the thermally insulated interior 4 of the ice maker 1 and arranging the first ozone generator 14 outside the thermally insulated interior 4 of the ice maker 1 and coupling the first air inlet 16 of the first ozone generator 14 and the first air outlet 17 of the first ozone generator 14 with the at least one aperture 32 in the ice maker 1 between the outside of the ice making section 2 of the thermally insulated interior 4 of the ice maker 1 and the ice making section 2 of the thermally insulated interior 4 of the ice maker 1.

This preferred embodiment of the method includes preferably, but not necessarily, the steps of providing at least one aperture 32 in the ice maker 1 between the outside of the ice storage section 3 of the thermally insulated interior 4 of the ice maker 1 and the ice storage section 3 of the thermally insulated interior 4 of the ice maker 1 and arranging the second ozone generator 19 outside the thermally insulated interior 4 of the ice maker 1 and coupling the second air inlet 21 of the second ozone generator 19 and the second air outlet 22 of the second ozone generator 19 with the at least one aperture 32 in the ice maker 1 between the outside of the ice storage section 3 of the thermally insulated interior 4 of the ice maker 1 and the ice storage section 3 of the thermally insulated interior 4 of the ice maker 1. This preferred embodiment of the method includes preferably, but not necessarily, the steps of providing the first air inlet 17 of the first ozone generator 14 with a third tube 33 for leading air into the first housing 15 of the first ozone generator 14 and providing the first air outlet 17 of the first ozone generator 14 with a fourth tube 34 for feeding air from the first housing 15 of the first ozone generator 14 and fitting the third tube 33 and the fourth tube 34 in the at least one aperture 32 provided between the outside of the ice making section 2 of the thermally insulated interior 4 of the ice maker 1 and the ice making section 2 of the thermally insulated interior 4 of the ice maker 1.

This preferred embodiment of the method includes preferably, but not necessarily, the steps of providing the second air inlet 21 of the second ozone generator 19 with a fifth tube 35 for leading air into the second housing 20 of the second ozone generator 19 and providing the second air outlet 22 of the second ozone generator 19 with a sixth tube 36 for feeding air from the second housing 20 of the second ozone generator 19 and fitting the fifth tube 35 and the sixth tube 36 in the at least one aperture 32 provided between the outside of the ice storage section 3 of the thermally insulated interior 4 of the ice maker 1 and the ice storage section 3 of the thermally insulated interior 4 of the ice maker 1.

It is obvious for a person skilled in the art that as techniques develop, the basic idea of the invention can be implemented in various ways. Consequently, the invention and the embodiments thereof are not confined to the above examples but can vary within the scope of the claims.

What is claimed is:

1. An ice bin for storing ice, comprising:
an ozone generator for generating ozone,
the ozone generator including a housing having an air inlet and an air outlet,
wherein the air inlet of the housing of the ozone generator is functionally connected to the air outlet of the housing of the ozone generator for allowing air to flow through the housing,
the ozone generator further including ozone generating means in the housing for generating ozone in air flowing through the housing,
wherein the air outlet of the housing of the ozone generator is functionally connected to a thermally insulated interior of the ice bin for feeding air containing ozone from the housing of the ozone generator to the thermally insulated interior of the ice bin, and
the air inlet of the housing of the ozone generator is functionally connected to the thermally insulated interior of the ice bin for receiving air from the thermally insulated interior of the ice bin.

2. The ice bin of claim 1,
wherein the ozone generator is placed outside the thermally insulated interior of the ice bin,
the ice bin is provided with at least one aperture provided between the outside of the thermally insulated interior of the ice bin and the thermally insulated interior of the ice bin, and
the air outlet of the housing of the ozone generator and the air inlet of the housing of the ozone generator are functionally connected with the at least one aperture of the ice maker for feeding air containing ozone from the ozone generator into the thermally insulated interior of the ice bin and for receiving air from the thermally insulated interior of the ice bin with the ozone generator.

3. The ice bin of claim 2,
wherein the air inlet of the ozone generator comprises a first tube for leading air into the housing of the ozone generator and the air outlet of the ozone generator comprises a second tube for feeding air from the housing of the ozone generator, and
the first tube and the second tube are fitted in the at least one aperture of the ice bin.

4. An ice maker, comprising:
an ozone generator for generating ozone,
the ozone generator including a housing having an air inlet and an air outlet,
wherein the air inlet of the housing of the ozone generator is functionally connected to the air outlet of the housing of the ozone generator for allowing air to flow through the housing,
the ozone generator further including ozone generating means in the housing for generating ozone in air flowing through the housing,
wherein the air outlet of the housing of the ozone generator is functionally connected to a thermally insulated interior of the ice maker for feeding air containing ozone from the housing of the ozone generator to the thermally insulated interior of the ice maker, and
the air inlet of the housing of the ozone generator is functionally connected to the thermally insulated interior of the ice maker for receiving air from the thermally insulated interior of the ice maker.

5. The ice maker of claim 4,
wherein the thermally insulated interior of the ice maker comprises an ice making section comprising ice making means for making ice,
the thermally insulated interior of the ice maker comprises an ice storage section for storing ice, the ice storage section being configured to receive ice from an ice making section of the thermally insulated interior of the ice maker, and
at least one of the air inlet and the air outlet of the ozone generator is functionally connected with the ice making section.

6. The ice maker of claim 5,
wherein the ozone generator is placed outside the thermally insulated interior of the ice maker,
the ice maker is provided with at least one aperture provided between the outside of the ice making section of the thermally insulated interior of the ice maker and the ice making section of the thermally insulated interior of the ice maker, and
at least one of the air outlet of the ozone generator and the air inlet of the ozone generator is functionally connected with the at least one aperture provided between the outside of the ice making section of the thermally insulated interior of the ice maker and the ice making section of the thermally insulated interior of the ice maker for feeding air containing ozone from the ozone generator into the ice making section of the thermally insulated interior of the ice maker, or respectively, for receiving air from the ice making section of the thermally insulated interior of the ice maker with the ozone generator.

7. The ice maker of claim 6,
wherein the air inlet of the ozone generator comprises a first tube for leading air into the housing of the ozone generator and the air outlet of the ozone generator comprises a second tube for feeding air from the housing of the ozone generator, and
at least one of the first tube and the second tube are fitted in the at least one aperture provided between the outside of the ice making section of the thermally insulated interior of the ice maker and the ice making section of the thermally insulated interior of the ice maker.

8. The ice maker of claim 4,
wherein the thermally insulated interior of the ice maker comprises an ice making section comprising ice making means for making ice,
the thermally insulated interior of the ice maker comprises an ice storage section for storing ice, the ice storage section being configured to receive ice from an ice making section of the thermally insulated interior of the ice maker
the air inlet is functionally connected with the ice making section for receiving air from the ice making section, and
the air outlet is functionally connected with the ice making section for feeding air containing ozone from the ozone generator to the ice making section.

9. The ice maker of claim 8,
wherein the ozone generator is placed outside the thermally insulated interior of the ice maker,
the ice maker being provided with at least one aperture provided between the outside of the ice making section of the thermally insulated interior of the ice maker and the ice making section of the thermally insulated interior of the ice maker, and
the air outlet of the housing of the ozone generator and the air inlet of the housing of the ozone generator are functionally connected with the at least one aperture provided between the outside of the ice making section of the thermally insulated interior of the ice maker and the ice making section of the thermally insulated interior of the ice maker for feeding air containing ozone from the ozone generator into the ice making section of the thermally insulated interior of the ice maker and for receiving air from the ice making section of the thermally insulated interior of the ice maker with the ozone generator.

10. The ice maker of claim 9,
wherein the air inlet of the ozone generator comprises a first tube for leading air into the housing of the ozone generator and wherein the air outlet of the ozone generator comprises a second tube for feeding air from the housing of the ozone generator, and
the first tube and the second tube are fitted in the at least one aperture provided between the outside of the ice making section of the thermally insulated interior of the ice maker and the ice making section of the thermally insulated interior of the ice maker.

11. The ice maker of claim 4,
wherein the thermally insulated interior of the ice maker comprises an ice making section comprising ice making means for making ice,
the thermally insulated interior of the ice maker comprises an ice storage section for storing ice, the ice storage section being configured to receive ice from an ice making section of the thermally insulated interior of the ice maker, and
at least one of the air inlet and the air outlet of the ozone generator is functionally connected with the ice storage section.

12. The ice maker of claim 11,
wherein the ozone generator is placed outside the thermally insulated interior of the ice maker,
the ice maker being provided with at least one aperture provided between the outside of the ice storage section of the thermally insulated interior of the ice maker and the ice storage section of the thermally insulated interior of the ice maker, and
at least one of the air outlet of the ozone generator and the air inlet of the ozone generator are functionally connected with the at least one aperture provided between the outside of the ice storage section of the thermally insulated interior of the ice maker and the ice storage section of the thermally insulated interior of the ice maker for feeding air containing ozone from the ozone generator into the ice storage section of the thermally insulated interior of the ice maker, or respectively, for receiving air from the ice storage section of the thermally insulated interior of the ice maker with the ozone generator.

13. The ice maker of claim 12,
wherein the air inlet of the ozone generator comprises a first tube for leading air into the housing of the ozone generator and wherein the air outlet of the ozone generator comprises a second tube for feeding air from the housing of the ozone generator, and
the first tube and the second tube are fitted in the at least one aperture provided between the outside of the ice storage section of the thermally insulated interior of the ice maker and the ice storage section of the thermally insulated interior of the ice maker.

14. The ice maker of claim 4,
wherein the thermally insulated interior of the ice maker comprises an ice making section comprising ice making means for making ice,
the thermally insulated interior of the ice maker comprises an ice storage section for storing ice, the ice storage section being configured to receive ice from an ice making section of the thermally insulated interior of the ice maker,
the air inlet is functionally connected with the ice storage section for receiving air from the ice storage section, and
the air outlet is functionally connected with the ice storage section for feeding air containing ozone from the ozone generator into the ice storage section.

15. The ice maker of claim 14,
wherein the ozone generator is placed outside the thermally insulated interior of the ice maker,
wherein the ice maker being provided with at least one aperture provided between the outside of the ice storage section of the thermally insulated interior of the ice maker and the ice storage section of the thermally insulated interior of the ice maker, and
the air outlet of the ozone generator and the air inlet of the ozone generator are functionally connected with the at least one aperture provided between the outside of the ice storage section of the thermally insulated interior of the ice maker and the ice storage section of the thermally insulated interior of the ice maker for feeding air containing ozone from the ozone generator into the ice storage section of the thermally insulated interior of the ice maker and for receiving air from the ice storage section of the thermally insulated interior of the ice maker with the ozone generator.

16. The ice maker of claim 15,
wherein the air inlet of the ozone generator comprises a first tube for leading air into the housing of the ozone generator and the air outlet of the ozone generator comprises a second tube for feeding air from the housing of the ozone generator, and
the first tube and the second tube are fitted in the at least one aperture provided between the outside of the ice storage section of the thermally insulated interior of the ice maker and the ice storage section of the thermally insulated interior of the ice maker.

17. A method for cleaning an ice bin for storing ice, comprising the steps of:
providing an ozone generator for generating ozone, the ozone generator comprising a housing having an air inlet and an air outlet, wherein the air inlet of the housing of the ozone generator is functionally connected to the air outlet of the housing of the ozone generator for allowing air to flow through the housing, and ozone generating means in the housing for generating ozone in the air flowing through the housing,
coupling the air inlet of the housing of the ozone generator to a thermally insulated interior of the ice bin,
coupling the air outlet of the housing to an thermally insulated interior of the ice maker,
operating the ozone generator to cause air to flow from the thermally insulated interior of the ice bin through the housing of the ozone generator and back to the thermally insulated interior of the ice bin, and
operating the ozone generating means inside the housing for generating ozone in the air flowing through the housing.

18. The method of claim 17, and further including the steps of:
providing at least one aperture in the ice bin between the outside of the thermally insulated interior of the ice bin and the thermally insulated interior of the ice bin,
arranging the ozone generator outside the thermally insulated interior of the ice bin, and
coupling the air inlet of the ozone generator and the air outlet of the ozone generator with the at least one aperture in the ice bin between the outside of the thermally insulated interior of the ice bin and the thermally insulated interior of the ice bin.

19. The method of claim 18, and further including the steps of:
providing the air inlet of the ozone generator with a first tube for leading air into the housing of the ozone generator,
providing the air outlet of the ozone generator with a second tube for feeding air from the housing of the ozone generator, and
arranging the first tube and the second tube in the at least one aperture of the ice bin so that the ozone generator is placed outside the thermally insulated interior of the ice bin and the first tube and the second tube are functionally connected with the thermally insulated interior of the ice maker.

20. A method for cleaning an ice maker, the method comprising the steps of:
providing an ozone generator for generating ozone, the ozone generator comprising a housing having an air inlet and an air outlet, wherein the air inlet of the housing of the ozone generator is functionally connected to the air outlet of the housing of the ozone generator for allowing air to flow through the housing, and ozone generating means in the housing for generating ozone in the air flowing through the housing,
coupling the air inlet of the housing of the ozone generator to an thermally insulated interior of the ice maker,
coupling the air outlet of the housing to an thermally insulated interior of the ice maker,
operating the ozone generator to cause air to flow from the interior of the ice maker through the housing of the ozone generator and back to the interior of the ice maker, and
operating the ozone generating means inside the housing for generating ozone in the air flowing through the housing.

21. The method of claim 20, further including the step of coupling the air inlet of the ozone generator to an ice making section of the thermally insulated interior of the ice maker.

22. The method of claim 21, further including the steps of:
providing at least one aperture in the ice maker between the outside of the ice making section of the thermally insulated interior of the ice maker and the ice making section of the thermally insulated interior of the ice maker,
arranging the ozone generator outside the thermally insulated interior of the ice maker, and
coupling the air inlet of the ozone generator and the air outlet of the ozone generator with the at least one aperture in the ice maker between the outside of the ice making section of the thermally insulated interior of the ice maker and the ice making section of the thermally insulated interior of the ice maker.

23. The method of claim 22, further including the steps of:
providing the air inlet of the ozone generator with a first tube for leading air into the housing of the ozone generator,
providing the air outlet of the ozone generator with a second tube for feeding air from the housing of the ozone generator, and
arranging the first tube and the second tube in the at least one aperture of the ice maker so that the ozone generator is placed outside the ice making section of the thermally insulated interior of the ice maker and the first tube and the second tube are functionally connected with the ice making section of the thermally insulated interior of the ice maker.

24. The method of claim 20, further including the step of coupling the air inlet of the ozone generator to an ice storage section of the thermally insulated interior of the ice maker, the ice storage section being configured to receive ice from an ice making section of the thermally insulated interior of the ice maker.

25. The method of claim 24, further including the steps of:
providing at least one aperture in the ice maker between the outside of the ice storage section of the thermally insulated interior of the ice maker and the ice storage section of the thermally insulated interior of the ice maker,
arranging the ozone generator outside the thermally insulated interior of the ice maker, and
coupling the air inlet of the ozone generator and the air outlet of the ozone generator with the at least one aperture in the ice maker between the outside of the ice storage section of the thermally insulated interior of the ice maker and the ice storage section of the thermally insulated interior of the ice maker.

26. The method of claim 25, further including the steps of:
providing the air inlet of the ozone generator with a first tube for leading air into the housing of the ozone generator,
providing the air outlet of the ozone generator with a second tube for feeding air from the housing of the ozone generator, and
arranging the first tube and the second tube in the at least one aperture of the ice maker so that the ozone generator is placed outside the ice storage section of the thermally insulated interior of the ice maker and the first tube and the second tube are functionally connected with the ice storage section of the thermally insulated interior of the ice maker.
